(19) United States
(12) Patent Application Publication
(10) Pub. No.: US 2019/0287665 A1
(43) Pub. Date: Sep. 19, 2019

(54) TEMPLATE-BASED MEDICAL SUMMARY INTERFACE GENERATION SYSTEM

(71) Applicant: KONINKLIJKE PHILIPS N.V., EINDHOVEN (NL)

(72) Inventors: Thomas Andre Forsberg, Hayward, CA (US); Merlijn Sevenster, Haarlem (NL)

(21) Appl. No.: 16/336,222

(22) PCT Filed: Oct. 6, 2017
(86) PCT No.: PCT/EP2017/075571

§ 371 Date: Mar. 25, 2019

Related U.S. Application Data
(60) Provisional application No. 62/404,786, filed on Oct. 6, 2016.

(51) Int. Cl. G16H 15/00; G16H 10/60; G16H 50/30

(52) U.S. Cl. CPC G16H 15/00; G16H 50/30; G16H 10/60

(57) ABSTRACT

The present disclosure pertains to a medical summary interface view generation system. Medical reports are often verbose, and a specific user may only be interested in a fraction of the information conveyed in the report. Typical generation of a report summary is time consuming and laborious. The present system eases the creation of report summaries by automatically emphasizing and/or de-emphasizing items in a view of a medical report that might be of interest to the user according to pre-defined templates. This preserves control of what is included in the summary view while removing the laborious task of typing out a report summary from scratch.

### BACKGROUND

### 1. Field

The present disclosure pertains to a medical summary interface view generation system.

### 2. Description of the Related Art

Electronic medical report summaries are known. A report summary typically aggregates the main findings in a medical report and summarizes a patient's condition and salient findings from the various procedures performed on the patient in a few key words and/or sentences. These summaries are often created through manual point-and-click selection of predetermined diagnostic codes, noted in free text (e.g., handwriting and/or typing), and/or are transcribed from voice recordings. Moreover, creating medical summaries from medical reports are often difficult, time consuming, and laborious, for example, because the medical reports are often verbose. Further, a specific user may only be interested in a fraction of the information conveyed in a given medical report, which may be tedious for a user to locate due to the verbose nature of medical reports and/or other issues with medical reports.

### SUMMARY

Accordingly, one or more aspects of the present disclosure relate to a medical summary interface view generation system comprising one or more hardware processors and/or other components. The one or more hardware processors are configured by machine-readable instructions to: obtain an electronic medical report associated with a subject; parse and associate words and/or phrases used in the medical report based on medical ontology information; identify concepts in the medical report, the concepts associated with one or more medical conditions experienced by the subject, the concepts identified based on the parsed and associated words and/or phrases; facilitate entry and/or selection of a word and/or phrase of interest in the electronic medical report from a caregiver of the subject, the word and/or phrase of interest corresponding to a given one of the identified concepts; select an interface template based on the word and/or phrase of interest and the given concept, the selected interface template comprising a tuple from the medical report relevant to the caregiver; determine words and/or phrases in the electronic medical report in addition to the word and/or phrase of interest that correspond to the given concept to populate the selected interface template; populate the selected interface template with the word and/or phrase of interest, the additional words and/or phrases that correspond to the given concept, and other words and/or phrases; and cause, based on the populated interface template, the word and/or phrase of interest and the additional words and/or phrases to be visually emphasized in a view of the electronic medical report on a graphical user interface, while deemphasizing the other words and/or phrases in the same view of the electronic medical report on the graphical user interface.

Yet another aspect of the present disclosure relates to a method for generating a summary interface view of a medical report with a medical summary interface view generation system. The system comprises one or more hardware processors and/or other components. The method comprises: obtaining, with the one or more hardware processors, an electronic medical report associated with a subject; parsing and associating, with the one or more hardware processors, words and/or phrases used in the medical report based on medical ontology information; identifying, with the one or more hardware processors, concepts in the medical report, the concepts associated with one or more medical conditions experienced by the subject, the concepts identified based on the parsed and associated words and/or phrases; facilitating, with the one or more hardware processors, entry and/or selection of a word and/or phrase of interest in the electronic medical report from a caregiver of the subject, the word and/or phrase of interest corresponding to a given one of the identified concepts; selecting, with the one or more hardware processors, an interface template based on the word and/or phrase of interest and the given concept, the selected interface template comprising a tuple from the medical report relevant to the caregiver; determining, with the one or more hardware processors, words and/or phrases in the electronic medical report in addition to the word and/or phrase of interest that correspond to the given concept to populate the selected interface template; populating, with the one or more hardware processors, the selected interface template with the word and/or phrase of interest, the additional words and/or phrases that correspond to the given concept, and other words and/or phrases; and causing, with the one or more hardware processors, based on the populated interface template, the word and/or phrase of interest and the additional words and/or phrases to be visually emphasized in a view of the electronic medical report on a graphical user interface, while deemphasizing the other words and/or phrases in the same view of the electronic medical report on the graphical user interface.

Still another aspect of present disclosure relates to a system for generating a summary interface view of a medical report. The system comprises: means for obtaining an electronic medical report associated with a subject; means for parsing and associating words and/or phrases used in the medical report based on medical ontology information; means for identifying concepts in the medical report, the concepts associated with one or more medical conditions experienced by the subject, the concepts identified based on the parsed and associated words and/or phrases; means for facilitating entry and/or selection of a word and/or phrase of interest in the electronic medical report from a caregiver of the subject, the word and/or phrase of interest corresponding to a given one of the identified concepts; means for selecting an interface template based on the word and/or phrase of interest and the given concept, the selected interface template comprising a tuple from the medical report relevant to the caregiver; means for determining words and/or phrases in the electronic medical report in addition to the word and/or phrase of interest that correspond to the given concept to populate the selected interface template; means for populating the selected interface template with the word and/or phrase of interest, the additional words and/or phrases that correspond to the given concept, and other words and/or phrases; and means for causing based on the populated interface template, the word and/or phrase of interest and the additional words and/or phrases to be visually emphasized in a view of the electronic medical report on a graphical user interface, while deemphasizing the other words and/or phrases in the same view of the electronic medical report on the graphical user interface.

These and other objects, features, and characteristics of the present disclosure, as well as the methods of operation and functions of the related elements of structure and the combination of parts and economies of manufacture, will become more apparent upon consideration of the following description and the appended claims with reference to the accompanying drawings, all of which form a part of this specification, wherein like reference numerals designate corresponding parts in the various figures. It is to be expressly understood, however, that the drawings are for the purpose of illustration and description only and are not intended as a definition of the limits of the disclosure.

### BRIEF DESCRIPTION OF THE DRAWINGS

FIG. 1 is a schematic illustration of a medical summary interface view generation system.

FIG. 2 illustrates views of a graphical user interface displayed to a caregiver.

FIG. 3 illustrates view of the graphical user interface generated based on selection of multiple phrases of interest.

FIG. 4 illustrates a method for generating a summary interface view of a medical report with a medical summary interface view generation system.

### DETAILED DESCRIPTION OF EXEMPLARY EMBODIMENTS

As used herein, the singular form of “a”, “an”, and “the” include plural references unless the context clearly dictates otherwise. As used herein, the statement that two or more parts or components are “coupled” shall mean that the parts are joined or operate together either directly or indirectly, i.e., through one or more intermediate parts or components, so long as a link occurs. As used herein, “directly coupled” means that two elements are directly in contact with each other. As used herein, “fixedly coupled” or “fixed” means that two components are coupled so as to move as one while maintaining a constant orientation relative to each other.

As used herein, the word “unitary” means a component is created as a single piece or unit. That is, a component that includes pieces that are created separately and then coupled together as a unit is not a “unitary” component or body. As employed herein, the statement that two or more parts or components “engage” one another shall mean that the parts exert a force against one another either directly or through one or more intermediate parts or components. As employed herein, the term “number” shall mean one or an integer greater than one (i.e., a plurality).

Directional phrases used herein, such as, for example and without limitation, top, bottom, left, right, upper, lower, front, back, and derivatives thereof, relate to the orientation of the elements shown in the drawings and are not limiting upon the claims unless expressly recited therein.

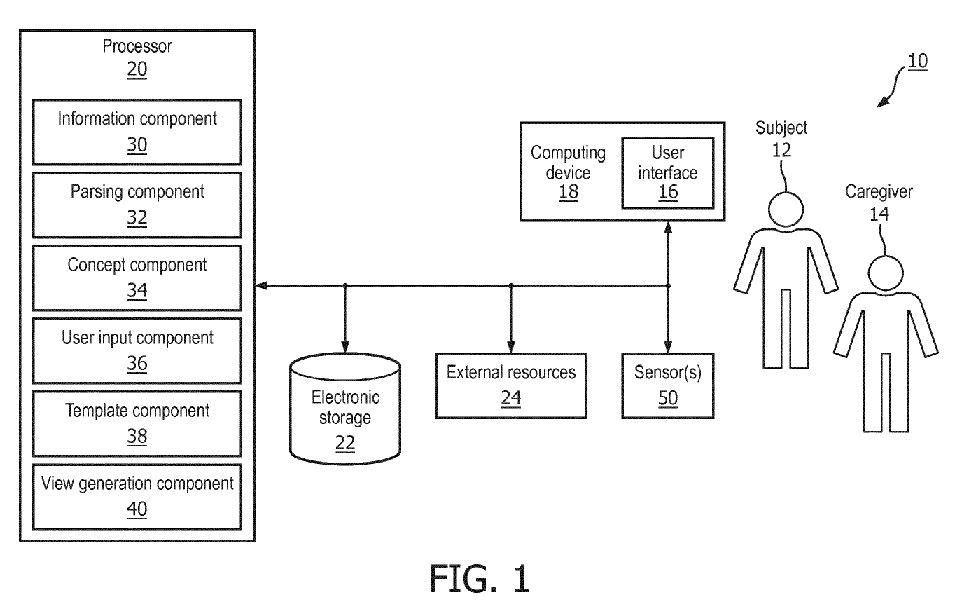

FIG. 1 is a schematic illustration of a medical summary interface view generation system 10. Medical reports document and maintain findings obtained during medical exams and procedures. Medical reports are thorough in their description yet concise in their message. There are a wide range of different types and styles of medical reports that depend on the medical domain, (e.g., oncology, radiology, cardiology etc.), the health care provider and/or other vendors, modalities (e.g., ultrasound, MRI, etc.), and/or other factors. Such reports are often verbose in their description, and often a busy caregiver (e.g., clinician, doctor, nurse, care facility staff, family member, friend, etc.) would prefer an “at-a-glance” overview of a subject's condition.

A specific caregiver 14 may only be interested in a fraction of the information conveyed in a report, for example, as a reminder before, during, and/or after a medical intervention, treatment planning, and/or at other times. The information of interest to caregiver 14 is predictable, in the sense that the combination of information relevant to a given medical intervention, treatment plan, and/or other task is likely known because such tasks have been previously performed by the same and/or other caregivers 14 on the same and/or similar subjects 12. For example, for a caregiver 14 about to perform a joint replacement surgery on a subject 12, it is generally predictable that caregiver 14 would be interested x-ray images of subject 12 and prior procedures performed on subject 12 in or near the location of the current surgery, but less concerned about bloodwork, pathology test results, medications taken, and/or other similar information included in a report and/or other medical records associated with subject 12. As a second example, a caregiver 14 counseling a subject 12 prior to chemo therapy treatment is likely interested in the bloodwork, pathology test results, medications taken, and medical conditions experienced by subject 12, and less interested in x-rays and similar information in reports and/or other medical records. System 10 is configured to assist caregiver 14 in collecting the predictable combinations of information of interest from medical reports for a subject 12 (e.g., as described in the examples above), and to display the information of interest in a summary view of the report for easy digestion by caregiver 14. System 10 is configured such that the summary view of the report including the extracted combinations of information of interest to caregiver 14 may be shared with external resources (e.g., other computing systems). In some embodiments, system 10 comprises a computing device 18, one or more processors 20, electronic storage 22, external resources 24, sensors 50, and/or other components.

Computing device 18 is configured to provide an interface between caregiver 14 and system 10. In some embodiments, computing device 18 is associated with caregiver 14 and/or other users. Computing device 18 is configured to provide information to and/or receive information from caregiver 14 and/or other users. Computing device 18 includes a user interface 16 and/or other components. User interface 16 is configured to receive entry and/or selection of information related to an identity of caregiver 14, words and/or phrases of interest to caregiver 14 in a medical report associated with subject 12, and/or other information. User interface 16 is configured to present caregiver 14 and/or other users with one or more summary views of the medical report. In some embodiments, user interface 16 includes a plurality of separate interfaces associated with computing device 18, processor 20 and/or other components of system 10, for example.

In some embodiments, computing device 18 is configured to provide user interface 16, processing capabilities, databases, and/or electronic storage to system 10. As such, computing device 18 may include processors 20, electronic storage 22, external resources 24, and/or other components of system 10. In some embodiments, computing device 18 is connected to a network (e.g., the internet). In some embodiments, computing device 18 does not include processor 20, electronic storage 22, external resources 24, and/or other components of system 10, but instead communicates with these components via the network. For example, processor 20, electronic storage 22, and external resources 24, and/or other components of system 10 may be located in a “public cloud” or “private cloud” (hereinafter “cloud”). Computing device 18 is for example a dumb device that receives information stored and processed in the public or private cloud. The connection to the network may be wireless or wired. For example, processor 20 may be located in a remote server and may wirelessly cause display of the summary views of the medical report via user interface 16 to caregiver 14 on computing device 18. In some embodiments, computing device 18 is a laptop, a desktop computer, a smartphone, a tablet computer, and/or other computing devices.

Examples of interface devices suitable for inclusion in user interface 16 include a touch screen, a keypad, touch sensitive and/or physical buttons, switches, a keyboard, knobs, levers, a display, speakers, a microphone, an indicator light, an audible alarm, a printer, and/or other interface devices. The present disclosure also contemplates that computing device 18 includes a removable storage interface. In this example, information may be loaded into computing device 18 from removable storage (e.g., a smart card, a flash drive, a removable disk) that enables caregiver 14 and/or other users to customize the implementation of computing device 18. Other exemplary input devices and techniques adapted for use with computing device 18 and/or user interface 16 include, but are not limited to, an RS-232 port, RF link, an IR link, a modem (telephone, cable, etc.) and/or other devices.

Processor 20 is configured to provide information processing capabilities in system 10. As such, processor 20 may comprise one or more of a digital processor, an analog processor, a digital circuit designed to process information, an analog circuit designed to process information, a state machine, and/or other mechanisms for electronically processing information. Although processor 20 is shown in FIG. 1 as a single entity, this is for illustrative purposes only. In some embodiments, processor 20 may comprise a plurality of processing units. These processing units may be physically located within the same device (e.g., a server), or processor 20 may represent processing functionality of a plurality of devices operating in coordination (e.g., one or more servers, computing device 18, devices that are part of external resources 24, electronic storage 22, and/or other devices.)

In some embodiments, processor 20, external resources 24, computing device 18, electronic storage 22, and/or other components may be operatively linked via one or more electronic communication links. For example, such electronic communication links may be established, at least in part, via a network such as the Internet, and/or other networks. It will be appreciated that this is not intended to be limiting, and that the scope of this disclosure includes embodiments in which these components may be operatively linked via some other communication media. In some embodiments, processor 20 is configured to communicate with external resources 24, computing device 18, electronic storage 22, and/or other components according to a client/server architecture, a peer-to-peer architecture, and/or other architectures.

As shown in FIG. 1, processor 20 is configured via machine-readable instructions to execute one or more computer program components. The one or more computer program components may comprise one or more of an information component 30, a parsing component 32, a concept component 34, a user input component 36, a template component 38, a view generation component 40, and/or other components. Processor 20 may be configured to execute components 30, 32, 34, 36, 38, and/or 40 by software; hardware; firmware; some combination of software, hardware, and/or firmware; and/or other mechanisms for configuring processing capabilities on processor 20.

It should be appreciated that although components 30, 32, 34, 36, 38, and 40 are illustrated in FIG. 1 as being co-located within a single processing unit, in embodiments in which processor 20 comprises multiple processing units, one or more of components 30, 32, 34, 36, 38, and/or 40 may be located remotely from the other components. The description of the functionality provided by the different components 30, 32, 34, 36, 38, and/or 40 described below is for illustrative purposes, and is not intended to be limiting, as any of components 30, 32, 34, 36, 38, and/or 40 may provide more or less functionality than is described. For example, one or more of components 30, 32, 34, 36, 38, and/or 40 may be eliminated, and some or all of its functionality may be provided by other components 30, 32, 34, 36, 38, and/or 40. As another example, processor 20 may be configured to execute one or more additional components that may perform some or all of the functionality attributed below to one of components 30, 32, 34, 36, 38, and/or 40.

Information component 30 is configured to obtain an electronic medical report associated with subject 12 and/or other subjects. The electronic medical report may be and/or include descriptions of medical procedures performed on subject 12, medications taken by subject 12, tests performed on subject 12, test results for subject 12, diagnosis and/or descriptions of medical conditions experienced by subject 12, and/or other medical information related to subject 12. The electronic medical report may be obtained by information component 30 from electronic storage included in system 10 (e.g., in electronic storage 22), from external databases and/or servers that are part of external resources 24 (e.g., in an electronic medical records system associated with a health care provider), and/or from other sources.

Parsing component 32 is configured to parse and associate words and/or phrases used in the medical report. Parsing component 32 is configured to parse and associate the words and/or phrases used in the medical report based on medical ontology information and/or other information. In some embodiments, the medical ontology information includes linked words and/or phrases in an ontology database, definitions of words and/or phrases in the ontology database, and/or other information. In some embodiments, the medical ontology information is and/or includes a contextual knowledge database comprising an ontology of medical terms and their relationships. By way of a non-limiting example, existing medical ontologies such as SNOMED, RxNorm, OGMS, etc., may be included in the contextual knowledge database.

Concept component 34 is configured to identify concepts in the obtained medical report. In some embodiments, the concepts are associated with one or more medical conditions experienced by subject 12 and/or other topics. The concepts are identified based on the parsed and associated words and/or phrases, and/or other information. In some embodiments, concept component 34 is and/or includes a concept extraction engine configured to extract concepts in narrative text based on lexical descriptions of concepts in a contextual knowledge database and/or based on other information. In some embodiments, concept component 34 is and/or includes a concept extraction engine such as MetaMap and/or other concept extraction engines. In some embodiments, words and/or phrases of interest are associated with two or more concepts identified by concept component 34.

User input component 36 is configured to facilitate entry and/or selection by caregiver 14 (and/or other users) of word(s) and/or phrase(s) of interest in the medical report. The words and/or phrases of interest correspond to a given one of the identified concepts. User input component 36 is configured to facilitate entry and/or selection of the words and/or phrases of interest via user interface 16 of computing device 18 and/or other information entry and/or selection devices. For example, user input component 36 may be configured to highlight clickable (e.g., via a mouse, a touch screen, etc.) words and/or phrases (e.g., words and/or phrases that have been parsed and associated, and determined to be associated with one or more concepts) in the text of the electronic medical report in a view of a graphical user interface presented via display 16. As another example, user input component 36 may be configured to receive typed entry of words and/or phrases of interest via an entry field displayed to caregiver 14 in the graphical user interface on display 16. In some embodiments, user input component 36 is configured to facilitate direct entry and/or selection of one or more of the concepts identified by concept component 30. For example, user input component 36 may cause presentation on computing device 18 of a list of identified concepts for selection (e.g., via a mouse click) by caregiver 14.

In some embodiments, user input component 36 is configured to facilitate entry and/or selection of identity information from caregiver 14. Identity information from caregiver 14 may include information related to a caregiver type (e.g., a doctor, a nurse, a family member, a friend, etc.), a domain associated with a caregiver (e.g., radiology, cardiology, etc.), modalities (e.g., ultrasound, MRI, etc.) performed by the caregiver, information preferences of caregiver 14, display preferences of caregiver 14, templates (described below) associated with caregiver 14, and/or other information.

In some embodiments, user input component 36 is configured to recognize report parameters, such as modalities described in the report, patient history, report author, and/or other parameters. Such recognition may be based on the parsed and/or associated words from the medical report, the concepts determined by concept component 34, information entered and/or selected by caregiver 14 and/or other users as facilitated by user input component 36, and/or other information. In some embodiments, user input component 36 is configured to, based on the identity information, the recognized parameters and/or other information in the medical report, and/or other information, suggest words, phrases, and/or concepts for selection by caregiver 14 and/or other users. The suggested words, phrases, and/or concepts may be displayed to caregiver 14 via computing device 18, for example, and/or other computing devices.

Template component 38 is configured to select an interface template. A given interface template is a guide for which information should be subsequently displayed to caregiver 14 in a view of the electronic medical report (described below). An individual template is a combination of relevant informational elements (e.g., catheter device, anatomical location, etc.). The selected interface template comprises a tuple from the medical report relevant to caregiver 14 and/or other elements. In some embodiments, an interface template is a pre-defined tuple of ontology concepts that together form information that is relevant to caregiver 14 and/or other users. In some embodiments, the tuple of the interface template includes elements from the medical report including one or more of a medical device associated with the subject, a treatment location, a treatment technique, a procedure name, a disease associated with the subject, a disease severity indication, a date, a caregiver name, a caregiver type, a health status indication of the subject, a physiological measurement associated with the subject, an injury experienced by the subject, a lesion associated with the subject, a lesion type, and/or other elements.

In some embodiments, template component 38 is and/or includes a template selection engine configured to retrieve templates from a template database (e.g., electronic storage 22 and/or a database included in external resources 24). In some embodiments, templates stored in the template database are stored based on the concepts described above, a domain associated with a template (e.g., radiology, cardiology, etc.), a user (e.g., caregiver 14) associated with a template, and/or other factors. Template component 38 is configured to retrieve templates based on the concepts recognized by concept component 34, the word(s) and/or phrase(s) of interest entered and/or selected by caregiver 14, and/or other information. In some embodiments, the interface template is selected based on the individual word and/or phrase of interest, the concept(s), and an identity of caregiver 14. In some embodiments, template component 38 is configured to determine an identity of caregiver 14 based on the identity information obtained by user input component 36 via computing device 18. Template component 38 is configured to select a template based on the identity of caregiver 14 because, for example, the elements of information that are of interest to a radiologist (e.g., a caregiver with a first identity) often don't overlap with the elements of information that are of interest to a different type of doctor, a nurse, a family member, and/or another caregiver (e.g., a caregiver with a second identity).

In some embodiments, template component 38 is configured to cause display of two or more selectable template options for selection by caregiver 14. For example, responsive to template component 38 determining that two or more templates are associated with the words and/or phrases of interest, the identified concept, and/or the identity of caregiver 14, template component 38 may cause presentation of a list of possible templates for selection by caregiver 14. In some embodiments, instead of and/or in addition to waiting for selection of a template by caregiver 14, template component is configured to automatically populate and store (described below) views of the medical report for all of the possible templates in such embodiments.

Template component 38 is configured to determine words and/or phrases from the electronic medical report for populating the template. The words and/or phrases are from the electronic medical report and are in addition to the words and/or phrases of interest. The additional words and/or phrases also correspond to the given concept and/or concepts. The selected interface template is populated with the word and/or phrase of interest, the additionally determined words and/or phrases that correspond to the given concept, and other (unrelated) words and/or phrases. If words and/or phrases of interest are associated with two or more concepts, the template(s) related to any and/or both of the concepts may be selected and populated by template component 38.

In some embodiments, responsive to the receipt of the words and/or phrases of interest and the determination of a concept and/or concepts associated with those words and/or phrases of interest, template component 38 accesses the contextual knowledge database, and searches the context knowledge database to locate the concept. Template component 38 accesses a list (for example) of concepts used in the templates (e.g., “anatomy”, “device”, “diagnosis”, etc.). Template component 38 traverses the abstraction layers of the contextual knowledge database until a match is established between the transmitted concept and the templates' concepts (e.g., “pneumonia” and “disease”). In some embodiments, these abstraction steps are implemented based on pre-existing relationships between concepts. For example, template component 38 may start from the transmitted concept (e.g., “pneumonia”) and generalize along the “is-a” relation until it hits a concept that is used in one of the templates. Thus, the concept may be generalized until it reaches the root node of the ontology. Template component 38 is configured to return words and/or phrases associated with the identified concept and/or related concepts (e.g., “pneumonia” can potentially match “disease” and “anatomy” (lungs) in the database for populating the template.

In some embodiments, template component 38 is configured to populate templates based on contextual information retrieved from the medical report (e.g., the report parameters), relationships in the contextual knowledge database, the medical ontology information, the caregiver identity information, and/or other information. For example, a specific doctor (e.g., caregiver 14) may always select lesions suspicious for hepatocellular carcinoma in reports of type CT for inclusion in a report summary. Template component 38 may auto-fill the appropriate template(s) based on this information. The methods for such automatic population may include population based on conditional probabilities, population based on machine learning classification methods, and/or other population methods. In addition, these methods may determine proximity of a concept's phrase relative to the one or more previously selected phrases.

View generation component 40 is configured to cause visual emphasis of the words and/or phrases that populate the template, and/or de-emphasis of the other words and/or phrases in a view of the medical report. The emphasized words and/or phrases and the de-emphasized words and/or phrases are displayed in the same view of the electronic medical report. The view of the medical report is displayed in a graphical user interface (e.g., via user interface 16) to caregiver 14 via computing device 18. In this way, for example, the graphical user interface may be enhanced to provide caregiver 14 an “at-a-glance” overview of a subject's condition or otherwise facilitate viewing of the medical report (e.g., quicker or better review of the medical report). In some embodiments, view generation component 40 and/or the other components of processor 20 are configured such that after entry and/or selection of word(s) and/or phrase(s) of interest by caregiver 14 and/or other users, the visual emphasis/de-emphasis in the view of the medical report appear to occur in substantially real time to caregiver 14 and/or the other users. In some embodiments, view generation component 40 is configured to cause visual emphasis of the words and/or phrases that populate the template, without causing de-emphasis of the other words and/or phrases in the medical report. In some embodiments, view generation component 40 is configured to cause visual de-emphasis of the other words and/or phrases in the medical report, without causing emphasis of the words and/or phrases that populate the template.

In some embodiments, view generation component 40 is configured to electronically store the view of the medical report for electronic access by external computing systems. In addition, a short summary sentence based the populated template and/or summary view may be determined. This sentence may be stored with the summary view, for example. The view of the medical report and the short summary sentence are stored in a format that is accessible by the external computing systems. In some embodiments, the stored format is determined by view generation component 40 based on an application format required by the external computing systems. For example, in some embodiments, view generation component 40 is and/or includes an information broker that serves the information created by system 10 to third party applications (e.g., Intellispace Cardiovascular (ISCV) and/or any other software solution in which a user may read medical reports).

In some embodiments, view generation component 40 is configured to generate multiple different summary views of the electronic medical report. The multiple different summary views may be generated responsive to entry and/or selection of additional words and/or phrases of interest from the electronic medical report by caregiver 14 and/or other users; responsive to a plurality of templates being associated with the words and/or phrases of interest, the identified concept, and/or the identity of caregiver 14 (e.g., as described above); and/or other factors.

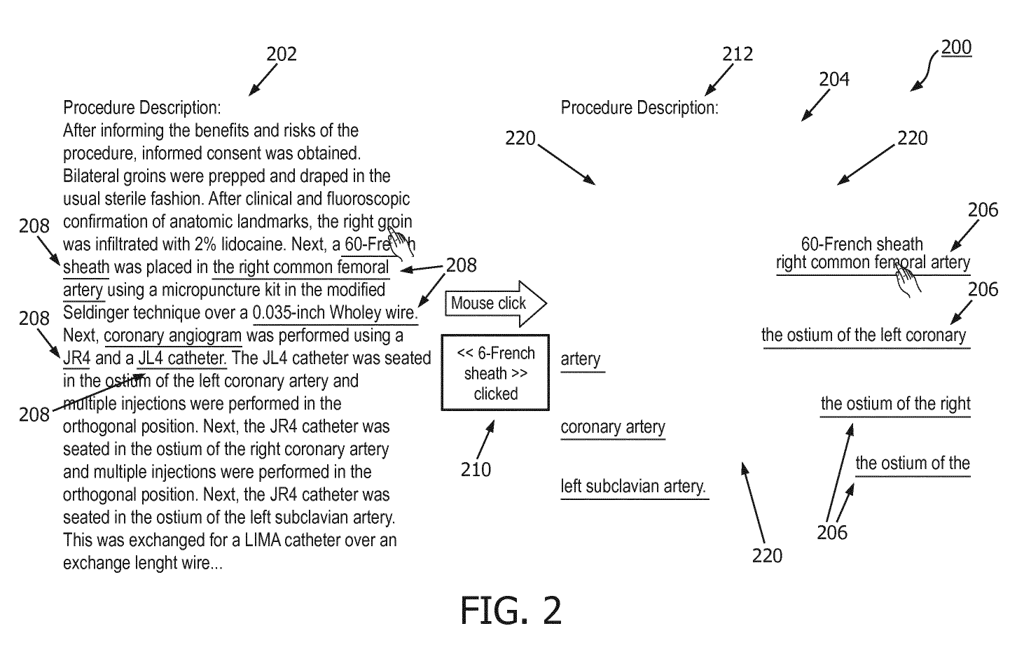

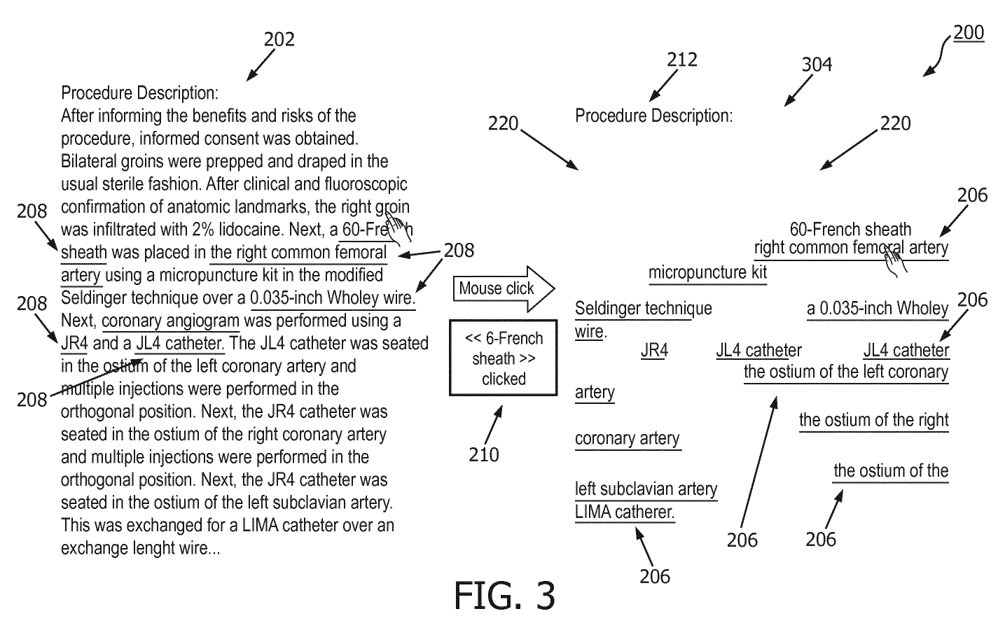

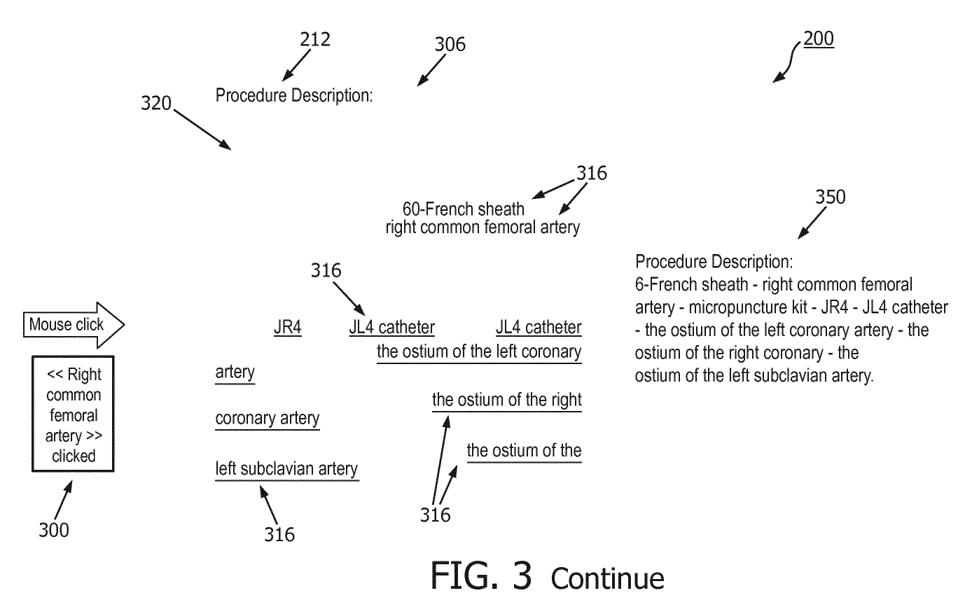

By way of a non-limiting example, FIG. 2 and FIG. 3 illustrate examples of the operations performed by system 10 (FIG. 1). FIG. 2 illustrates views 202, 204 of a graphical user interface 200 displayed (e.g., via user interface 16 shown in FIG. 1) to caregiver 14 (FIG. 1). FIG. 2 illustrates two views 202 and 204 of a portion (e.g., “Procedure Description”) of a medical report. View 202 illustrates parsed words and/or phrases 208 that were highlighted and made clickable (e.g., to facilitate selection of a phrase of interest as described above). View 204 displays the report with visually marked phrases 206 in the report from which concepts were extracted (e.g., as described above). In the example shown in FIG. 2, as a phrase is selected (clicked), one or more concepts associated with the selected phrase are determined. The phrase of interest and/or the one or more concepts are used to determine one or more templates for the summary view of the report. If multiple templates match the phrase of interest and/or the one or more concepts, a dropdown list (not shown in FIG. 2) may appear next to the phrase that was selected, and the user (e.g., caregiver 14) may select a specific template. The report formatting (e.g., highlighting, lowlighting, etc.) will be updated in the summary view 204 of the report based on the selected template.

For example, responsive to the selected phrase (e.g., “6-French sheath”) 210 being associated with a device concept, a template specifying device and location elements may be selected (e.g., by template component 38). System 10 is configured to highlight phrases 206 associated with devices and/or locations (e.g., 6-French sheath, femoral artery, ostia) and/or other associated words and/or phrases 212 (e.g., procedure description) in view 204 of the report and lowlight (e.g., de-emphasize) the other words and/or phrases 220.

FIG. 3 illustrates, responsive to the mouse click on the phrase of interest (e.g., “6-French sheath”) 210, selecting a second template specifying device, location, tool, and technique elements. As such, additional phrases relative to view 204 in FIG. 2 are highlighted in view 304. FIG. 3 illustrates further selection 300 of an additional phrase of interest (e.g., “Right common femoral artery”) from the highlighted phrases 206 shown in view 304. The template selection and population process is repeated again (this time based on the selections of “6-French sheath” and “Right common femoral artery”) and system 10 returns view 306 with highlighted phases 316 and de-emphasized portions 320.

In some embodiments, responsive to multiple templates matching an identified concept and/or the words and/or phrases of interest, words and/or phrases that match elements in at least one of the possible templates may be highlighted. In such embodiments, system 10 (e.g., view generation component 40) is configured such that the highlighting is proportional in intensity to a number of templates with which the words and/or phrases are associated. If words and/or phrases of interest are associated with two or more concepts, the template(s) related to any and/or both of the concepts may be selected and populated by system 10. System 10 is configured such that the steps described above in relation to FIG. 2 and/or FIG. 3 are repeated until there are no more selectable words and/or phrases and/or a user (e.g., caregiver 14) manually terminates the process.

Once population of an individual template is completed, e.g., either explicitly indicated by caregiver 14 and/or implicitly if system 10 automatically populated possible alternative templates, the summary views of the medical report generated based on the populated temples are stored along with a copy of the report formatted in its original state, but showing the clickable words and/or phrases associated with identified concepts. In addition, short summary sentences 350 (shown in FIG. 3) based the populated templates and/or summary views may be determined. Sentences 350 may be stored with the summary views and stored such that they may be retrieved via external computing systems.

Returning to FIG. 1, electronic storage 22 comprises electronic storage media that electronically stores information. The electronic storage media of electronic storage 22 may comprise one or both of system storage that is provided integrally (i.e., substantially non-removable) with system 10 and/or removable storage that is removably connectable to system 10 via, for example, a port (e.g., a USB port, a firewire port, etc.) or a drive (e.g., a disk drive, etc.). Electronic storage 22 may be (in whole or in part) a separate component within system 10, or electronic storage 22 may be provided (in whole or in part) integrally with one or more other components of system 10 (e.g., computing device 18, processor 20, etc.). In some embodiments, electronic storage 22 may be located in a server together with processor 20, in a server that is part of external resources 24, in computing device 18 associated with subject 12, and/or in other locations. Electronic storage 22 may comprise one or more of optically readable storage media (e.g., optical disks, etc.), magnetically readable storage media (e.g., magnetic tape, magnetic hard drive, floppy drive, etc.), electrical charge-based storage media (e.g., EPROM, RAM, etc.), solid-state storage media (e.g., flash drive, etc.), and/or other electronically readable storage media. Electronic storage 22 may store software algorithms, information determined by processor 20 (e.g., summary views of the medical report(s)), information received via computing device 18 and/or user interface 16 and/or other external computing systems, information received from external resources 24, information received from sensors 50, and/or other information that enables system 10 to function as described herein. In some embodiments, electronic storage 22 is and/or includes a database storing pertinent data related to entries and/or selections by caregiver 14, identity information for caregiver 14, modalities described in the electronic report, concepts in the electronic report, templates, and/or other information.

External resources 24 include sources of information (e.g., databases, websites, etc.), external entities participating with system 10 (e.g., a medical records system of a health care provider that stores medical reports associated with subject 12), external home monitoring systems, one or more servers outside of system 10, a network (e.g., the internet), electronic storage, equipment related to Wi-Fi technology, equipment related to Bluetooth® technology, data entry devices, sensors, scanners, and/or other resources. In some implementations, some or all of the functionality attributed herein to external resources 24 may be provided by resources included in system 10. External resources 24 may be configured to communicate with processor 20, computing device 18, electronic storage 22, sensors 50, and/or other components of system 10 via wired and/or wireless connections, via a network (e.g., a local area network and/or the internet), via cellular technology, via Wi-Fi technology, and/or via other resources.

In some embodiments, system 10 includes sensors 50 and/or other components configured to generate information described in an electronic medical report. For example, sensors 50 are configured to generate output signals conveying information related to one or more vital signs of subject 12, and/or other medical information about subject 12. In some embodiments, sensors 50 include but are not limited to equipment used in hospitals, doctor's offices, and/or other medical facilities, in the home of subject 12, and/or in other locations to monitor vital signs and/or other physiological information (e.g., pulse rate monitors, blood pressure monitors, blood oxygenation monitors, glucose monitors, weight scales, thermometers, electrocardiogram (EKG) equipment, childbirth labor contraction monitors, etc.), test equipment (e.g., imaging equipment such as an MRI and/or an x-ray machine, an ultrasound, electroencephalogram (EEG) equipment, etc.), equipment for treating subject 12 (e.g., respirators/ventilators, light therapy devices, etc.), devices for entering and/or selecting information (e.g., desktop computers, laptop computers, tablet computers, smartphones, cameras, video equipment, etc.) associated with subject 12, and/or other devices. Sensors 50 may comprise one or more sensors that measure vital signs directly. For example, sensors 50 may include electrodes configured to detect electrical activity in the heart of subject 12, light based sensors coupled with a finger of subject 12 configured to detect a pulse rate and/or a blood chemistry (e.g., an oxygen level) of subject 12, a blood pressure cuff configured to facilitate determination of the blood pressure of subject 12, a thermometer coupled to subject 12, and/or other sensors. Sensors 50 may comprise one or more sensors that generate output signals conveying information related to vital signs of subject 12 indirectly. For example, one or more sensors 50 may include a camera configured to generate an output based on images of subject 12 (e.g., a heart rate of subject 12, respiration of subject 12, a temperature of subject 12, an oxygen saturation level of subject 12, and/or other characteristics of subject 12). Although sensors 50 are illustrated in FIG. 1 at a single location near subject 12, this is not intended to be limiting. Sensors 50 may include sensors disposed in a plurality of locations, such as for example, coupled (in a removable manner) with clothing of subject 12, worn by subject 12 (e.g., as a headband, wristband, a blood pressure cuff, a finger clip, etc.), positioned to point at subject 12 (e.g., a camera), temporarily held and/or supported by subject 12 (e.g., a thermometer), and/or in other locations.

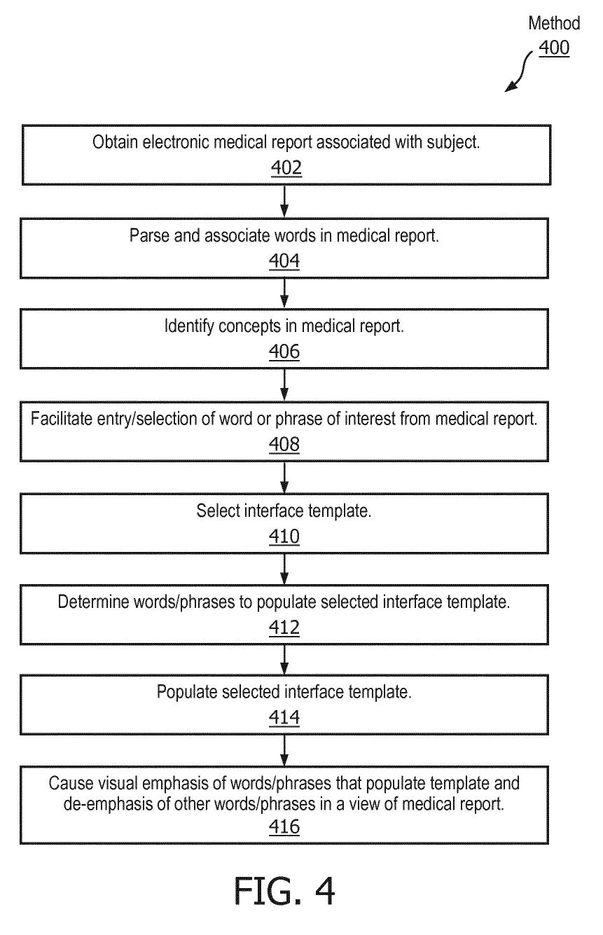

FIG. 4 illustrates a method 400 for generating a summary interface view of a medical report with a medical summary interface view generation system. The system comprises a computing device including a user interface, one or more hardware processors, and/or other components. The one or more hardware processors are configured by machine readable instructions to execute computer program components. The computer program components include an information component, a parsing component, a concept component, a user input component, a template component, a view generation component, and/or other components. The operations of method 400 presented below are intended to be illustrative. In some embodiments, method 400 may be accomplished with one or more additional operations not described, and/or without one or more of the operations discussed. Additionally, the order in which the operations of method 400 are illustrated in FIG. 4 and described below is not intended to be limiting.

In some embodiments, method 400 may be implemented in one or more processing devices (e.g., a digital processor, an analog processor, a digital circuit designed to process information, an analog circuit designed to process information, a state machine, and/or other mechanisms for electronically processing information). The one or more processing devices may include one or more devices executing some or all of the operations of method 400 in response to instructions stored electronically on an electronic storage medium. The one or more processing devices may include one or more devices configured through hardware, firmware, and/or software to be specifically designed for execution of one or more of the operations of method 400.

At an operation 402, an electronic medical report associated with a subject is obtained. In some embodiments, operation 402 is performed by a processor component the same as or similar to information component 30 (shown in FIG. 1 and described herein).

At an operation 404, words and/or phrases in the obtained medical report are parsed and/or associated. The parsing and/or associating is performed based on medical ontology information and/or other information. In some embodiments, the medical ontology information includes linked words and/or phrases in an otology database, definitions of words and/or phrases in the ontology database, and/or other information. In some embodiments, operation 404 is performed by a processor component the same as or similar to parsing component 32 (shown in FIG. 1 and described herein).

At an operation 406, concepts in the medical report are identified. In some embodiments, the concepts are associated with one or more medical conditions experienced by the subject and/or other topics. The concepts are identified based on the parsed and associated words and/or phrases, and/or other information. In some embodiments, operation 406 is performed by a processor component the same as or similar to concept component 34 (shown in FIG. 1 and described herein).

At an operation 408, entry and/or selection by a caregiver of words and/or phrases of interest in the medical report is facilitated. The words and/or phrases of interest correspond to a given one of the identified concepts. In some embodiments, operation 408 comprises facilitating entry and/or selection of identity information from the caregiver. In some embodiments, operation 408 is performed by a processor component the same as or similar to user input component 36 (shown in FIG. 1 and described herein).

At an operation 410, an interface template is selected. The interface template is selected based on the words and/or phrases of interest, the given concept, and/or other information. In some embodiments, the interface template is selected based on the individual words and/or phrases of interest, the given concept, and an identity of the caregiver. The selected interface template comprises a tuple from the medical report relevant to the caregiver and/or other elements. In some embodiments, the tuple of the interface template includes elements from the medical report including one or more of a medical device associated with the subject, a treatment location, a treatment technique, a procedure name, a disease associated with the subject, a disease severity indication, a date, a caregiver name, a caregiver type, a health status indication of the subject, a physiological measurement associated with the subject, an injury experienced by the subject, a lesion associated with the subject, a lesion type, and/or other elements. In some embodiments, operation 410 is performed by a processor component the same as or similar to template component 38 (shown in FIG. 1 and described herein).

At an operation 412, words and/or phrases for populating the template are determined. The words and/or phrases are from the electronic medical report and are in addition to the words and/or phrases of interest. The additional words and/or phrases also correspond to the given concept. In some embodiments, operation 412 is performed by a processor component the same as or similar to template component 38 (shown in FIG. 1 and described herein).

At an operation 414, the selected interface template is populated with the word and/or phrase of interest, the additionally determined words and/or phrases that correspond to the given concept, and other words and/or phrases. In some embodiments, operation 414 is performed by a processor component the same as or similar to template component 38 (shown in FIG. 1 and described herein).

At an operation 416, visual emphasis of the words and/or phrases that populate the template, and de-emphasis of the other words and/or phrases in a view of the medical report are caused. The emphasized word and/or phrases and the de-emphasized words and/or phrases are displayed in the same view of electronic medical report. The view of the medical report is displayed in a graphical user interface to the caregiver via a computing device. In some embodiments, operation 416 comprises electronically storing the view of the medical report for electronic access by external computing systems. In some embodiments, operation 416 comprises generating multiple different views of the electronic medical report. The multiple different views may be generated responsive to entry and/or selection of additional words and/or phrases of interest from the electronic medical report by the caregiver and/or a second caregiver, for example. In some embodiments, operation 416 is performed by a processor component the same as or similar to view generation component 40 (shown in FIG. 1 and described herein).

Although the description provided above provides detail for the purpose of illustration based on what is currently considered to be the most practical and preferred embodiments, it is to be understood that such detail is solely for that purpose and that the disclosure is not limited to the expressly disclosed embodiments, but, on the contrary, is intended to cover modifications and equivalent arrangements that are within the spirit and scope of the appended claims. For example, it is to be understood that the present disclosure contemplates that, to the extent possible, one or more features of any embodiment can be combined with one or more features of any other embodiment.

In the claims, any reference signs placed between parentheses shall not be construed as limiting the claim. The word “comprising” or “including” does not exclude the presence of elements or steps other than those listed in a claim. In a device claim enumerating several means, several of these means may be embodied by one and the same item of hardware. The word “a” or “an” preceding an element does not exclude the presence of a plurality of such elements. In any device claim enumerating several means, several of these means may be embodied by one and the same item of hardware. The mere fact that certain elements are recited in mutually different dependent claims does not indicate that these elements cannot be used in combination

1. A medical summary interface view generation system comprising one or more hardware processors configured by machine-readable instructions to:
obtain an electronic medical report associated with a subject;
parse and associate words and/or phrases used in the medical report based on medical ontology information;
identify concepts in the medical report, the concepts associated with one or more medical conditions experienced by the subject, the concepts identified based on the parsed and associated words and/or phrases;
facilitate entry and/or selection of a word and/or phrase of interest in the electronic medical report from a caregiver of the subject, the word and/or phrase of interest corresponding to a given one of the identified concepts;
select an interface template based on the word and/or phrase of interest and the given concept, the selected interface template comprising a tuple from the medical report relevant to the caregiver;
determine words and/or phrases in the electronic medical report in addition to the word and/or phrase of interest that correspond to the given concept to populate the selected interface template;
populate the selected interface template with the word and/or phrase of interest, the additional words and/or phrases that correspond to the given concept, and other words and/or phrases; and
cause, based on the populated interface template, the word and/or phrase of interest and the additional words and/or phrases to be visually emphasized in a view of the electronic medical report on a graphical user interface, while deemphasizing the other words and/or phrases in the same view of the electronic medical report on the graphical user interface.

2. The system of claim 1, wherein the one or more hardware processors are further configured to facilitate entry and/or selection of identity information from the caregiver; wherein the interface template is selected based on the individual word and/or phrase of interest, the given concept, and an identity of the caregiver; and wherein the tuple of the selected interface template comprises the elements of the medical report relevant to the caregiver.

3. The system of claim 1, wherein the one or more hardware processors are configured such that the tuple of the interface template includes elements from the medical report including one or more of a medical device associated with the subject, a treatment location, a treatment technique, a procedure name, a disease associated with the subject, a disease severity indication, a date, a caregiver name, a caregiver type, a health status indication of the subject, a physiological measurement associated with the subject, an injury experienced by the subject, a lesion associated with the subject, and/or a lesion type.

4. The system of claim 1, wherein the one or more hardware processors are configured to electronically store the view of the medical report for electronic access by external computing systems.

5. The system of claim 1, wherein the one or more hardware processors are configured to generate multiple different views of the electronic medical report, the multiple different views generated responsive to entry and/or selection of additional words and/or phrases of interest from the electronic medical report by the caregiver and/or a second caregiver.

6. The system of claim 1, further comprising a computing device associated with the caregiver that includes an interface, the one or more hardware processors further configured to cause display of the view of the electronic medical report on the graphical user interface to the caregiver via the interface of the computing device.

7. A method for generating a summary interface view of a medical report with a medical summary interface view generation system, the system comprising one or more hardware processors, the method comprising:
obtaining, with the one or more hardware processors, an electronic medical report associated with a subject;
parsing and associating, with the one or more hardware processors, words and/or phrases used in the medical report based on medical ontology information;
identifying, with the one or more hardware processors, concepts in the medical report, the concepts associated with one or more medical conditions experienced by the subject, the concepts identified based on the parsed and associated words and/or phrases;
facilitating, with the one or more hardware processors, entry and/or selection of a word and/or phrase of interest in the electronic medical report from a caregiver of the subject, the word and/or phrase of interest corresponding to a given one of the identified concepts;
selecting, with the one or more hardware processors, an interface template based on the word and/or phrase of interest and the given concept, the selected interface template comprising a tuple from the medical report relevant to the caregiver;
determining, with the one or more hardware processors, words and/or phrases in the electronic medical report in addition to the word and/or phrase of interest that correspond to the given concept to populate the selected interface template;
populating, with the one or more hardware processors, the selected interface template with the word and/or phrase of interest, the additional words and/or phrases that correspond to the given concept, and other words and/or phrases; and
causing, with the one or more hardware processors, based on the populated interface template, the word and/or phrase of interest and the additional words and/or phrases to be visually emphasized in a view of the electronic medical report on a graphical user interface, while deemphasizing the other words and/or phrases in the same view of the electronic medical report on the graphical user interface.

8. The method of claim 7, wherein the method further comprises facilitating, with the one or more hardware processors, entry and/or selection of identity information from the caregiver; wherein the interface template is selected based on the individual word and/or phrase of interest, the given concept, and an identity of the caregiver; and wherein the tuple of the selected interface template comprises the elements of the medical report relevant to the caregiver.

9. The method of claim 7, wherein the tuple of the interface template includes elements from the medical report including one or more of a medical device associated with the subject, a treatment location, a treatment technique, a procedure name, a disease associated with the subject, a disease severity indication, a date, a caregiver name, a caregiver type, a health status indication of the subject, a physiological measurement associated with the subject, an injury experienced by the subject, a lesion associated with the subject, and/or a lesion type.

10. The method of claim 7, further comprising electronically storing, with the one or more hardware processors, the view of the medical report for electronic access by external computing systems.

11. The method of claim 7, further comprising generating, with the one or more hardware processors, multiple different views of the electronic medical report, the multiple different views generated responsive to entry and/or selection of additional words and/or phrases of interest from the electronic medical report by the caregiver and/or a second caregiver.

12. The method of claim 7, further comprising a computing device associated with the caregiver that includes an interface, wherein the method further comprises causing, with the one or more hardware processors, display of the view of the electronic medical report on the graphical user interface to the caregiver via the interface of the computing device.

13. (canceled)

14. (canceled)

15. (canceled)

16. (canceled)

17. (canceled)

18. (canceled)
